(19) United States
(12) Patent Application Publication
(10) Pub. No.: US 2015/0039329 A1
(43) Pub. Date: Feb. 5, 2015

(54) METHOD FOR TRIGGERING AN ACTION OF A DEVICE AUTHORIZED BY A PAPER DOCUMENT

(71) Applicant: CareFusion Germany 326 GmbH, Kelberg (DE)

(72) Inventors: Michael Wirtz, Daun-Neunkirchen (DE); Jens Wiegland, Gimbsheim (DE); Andreas Kohlhof, Meudt (DE)

(21) Appl. No.: 14/380,368

(22) PCT Filed: Feb. 6, 2013
(86) PCT No.: PCT/EP2013/052270

§ 371 Date: Aug. 21, 2014

(30) Foreign Application Priority Data
Feb. 21, 2012 (EP) 12156381.1

(51) Int. Cl. G06Q 50/22; G06Q 10/08

(52) U.S. Cl. CPC G06Q 50/22; G06Q 10/08; G06Q 10/087
USPC 705/2

(57) ABSTRACT

The present disclosure relates to a method for triggering an action of a device authorized by a paper document. In the method, the paper document is placed in a receptacle, an application device applies a first unique identifier to the paper document and generates an electronic image of the paper document, wherein a second identifier is uniquely assigned to the first identifier, and an electronic document is generated from the electronic image and is linked to the second identifier. An image of the electronic document is then displayed to a second, remote location of a person authorized to sign and the electronic document is signed by the authorized. The electronic document, which is linked to the second identifier and signed, is then stored in a secure memory. Once this process is complete, the authorized action is executed by the device.

### BACKGROUND

The publication DE 103 25 961 A1 describes a method for computer-assisted output of a drug to a user by using an automated pharmacy storage system. The method known from this citation is derived from a known method for output of a drug in a pharmacy, in which drug packages are stored in an automated pharmacy shelf storage system. With this method, it is known, for example, that a pharmacist receives a prescription from a customer and makes the corresponding entries in an input device (for example, a computer keyboard) of the automated pharmacy shelf storage system with which the pharmacist specifies the drug packages specified on the prescription. Then, after retrieving the requested drug packages from the shelf, the pharmacist receives payment from the customer, if necessary, whereupon he transfers the drug packages to the customer.

### SUMMARY

The disclosure relates to a method for triggering an action of a device authorized by a paper document, wherein the action presupposes a unique assignment of a signature to the paper document.

The phrase “action of a device” should be understood in the context of this disclosure to refer to a mechanical or electromechanical action or a switching action of an electrical device, for example, switching on a therapy device (such as illumination with light of a predefined duration, for example) as well as output of a drug package or enabling of access to a drug package by releasing a lock. Such an action may be authorized by a paper document. A “paper document” should be understood here to refer to any printed and/or written document of a printable/writable material (for example, paper or a plastic film) but not an electronic document in the sense of a mere data file. “Authorization” means that the paper document should be a necessary prerequisite for executing a certain type of such action. If the action is the output of a drug package and/or the enabling of access by a person to a certain drug package, then the paper document may be a prescription, for example. The action of the device should presuppose a unique assignment of a signature to the paper document in order to be executed. In the case of a prescription for a drug output, this means, for example, that dispensing of the medication can be carried out only if a signature has been assigned to the prescription in a unique manner. Traditionally this is done by having the prescription signed by an authorized person (for example, a pharmacist) with a signature with which the pharmacist is expressing the fact that the medication indicated on the prescription has been dispensed. The present disclosure relates to automation of this procedure under the condition that the authorized person normally signing the paper document by a signature is not at the location where the paper document is located, and the action of the device is to be authorized so that direct signing of the paper document is impossible.

A method is disclosed which allows the pharmacist to carry out the output of the medication by remote control with the help of an automated drug storage system (also known as an automatic order-picking system) that is installed at a first location, namely the pharmacy, and also with the help of an output terminal connected to the storage system by means of a remote data transmission network (for example, the Internet) and to do so from a remote location (for example, a home workstation). In the method described in the document cited above, the customer first makes an entry by operating an input device on the output terminal (for example, by depressing a call button), this input indicating that the customer would like to request a drug. After receiving this input, a call signal is transmitted by means of a data remote transmission channel to a data communications unit set up at a remote site, transmitting it to a computer operated by a pharmacist, for example. Next a bidirectional data communication link is established between the pharmacist's computer and a computer at the output terminal. The two computers are each equipped with cameras, microphones, display screens and loudspeakers, so that a personal dialogue can be conducted between the pharmacist and the customer over this channel, with the pharmacist advising the customer within this context. In the course of this dialogue, the customer will enter a prescription into a prescription reader provided on the output terminal, for example. An image of the prescription thus read can then be transmitted to the pharmacist's computer. The pharmacist checks the prescription, then continues the dialogue with the customer and decides on the basis of appropriate entries which drug packages are to be transported out of the automated shelf storage system to the output terminal, where the drug packages to be output enter an output site to which the customer initially does not have access. Then the pharmacist checks on whether the correct drugs are at the output site. The pharmacist optionally also checks on whether the customer has made the payment required for output of the drug packages by means of payment devices located at the output terminal. The payment devices may include, for example, an automatic currency payment machine, a coin slot and/or a debit card. Finally, when all the requirements for output of the drug packages have been met, the pharmacist will enable access to the output site by remote control, so that the customer can pick up the drug packages. The citation above also discloses that detection of the drug packages to be output can be carried out, for example, by automated reading of the prescription in paper form, but the citation says nothing about the retention of the prescription form after output of the drugs.

When dispensing a prescription drug, a pharmacist may be required by law to note any changes on the prescription and to sign the prescription at the time of dispensing the drug packages identified on the prescription form. This is of course impossible with the known remote control dispensing system, because the pharmacist does not have direct access to the prescription form. It would therefore be desirable to create a method for remote dispensing of drug packages at a dispensing terminal, such that it will create the security required by law in dispensing prescription drugs, while allowing the pharmacist to assign his/her signature to the paper document (prescription) in a unique manner that cannot be manipulated subsequently.

Based on the known method described above, the object of the present disclosure is thus to create a method for triggering an action of a device authorized by a paper document, in which the action presupposes a unique assignment of a signature to a paper document, making it possible for the paper document and the authorized person providing the signature to be at separate locations.

In the method for triggering an action of a device authorized by a paper document in which the action presupposes a unique assignment of a signature to a paper document, the paper document is placed in a receptacle at a first location where the action is to be carried out. A first unique identifier is applied permanently to the paper document by an application device and an electronic image of the paper document is created, such that a second identifier is uniquely assigned to the first identifier and an electronic document is generated from the electronic image and is linked to the second identifier. The first location where the action is to be carried out is the location where an automatic drug dispensing machine is located, for example, or it may also be the location where a therapeutic measure is to be carried out by an automatic device. The first location is preferably the location at which a drug dispensing terminal is located, which is linked to an automated storage system, for example, an automatic order-picking system.

The first unique identifier applied permanently to the paper document is, for example, a printed number, a printed character code, barcode or 2D code or even an RFID transponder or memory chip glued to the paper document. Accordingly, the application device is, for example, a printer or even a gluing device for gluing a tag or an RFID chip to the paper document. The electronic image of the paper document is, for example, a graphic file, e.g., in the form of a PDF format. The second identifier uniquely assigned to the first identifier is a file name, for example, which includes a code of the first identifier. The second identifier may be linked to the electronic document, for example, by the fact that the electronic document is assigned a file name containing the code.

In a method described herein, an image of the electronic document is displayed on an output device of a data processing unit which is located at a second remote location and belongs to a person authorized for signing, and the electronic document is signed by the authorized person by means of a predetermined input into an input unit. The image of the electronic document may reflect the entire document or just a portion of the document. The output device is, for example, a computer display screen and the data processing unit located at the second remote site is a computer located at the pharmacist's home workstation, for example. The predefined input carried out by the authorized person to sign the electronic document electronically is the input of a PIN or a fingerprint, a retinal scan or reading of an electronic personal identification, for example. The respective input device is a computer keyboard, a chip card reader, a fingerprint scanner or a camera, for example.

The signed electronic document linked to the second identifier is stored in a secured memory. As soon as the signed electronic document has been stored, the authorized action is carried out by the device.

A method described herein replaces the application of a manual signature on a paper document, which is the prerequisite for execution of the authorized action by the device, by permanent application of the first identifier to the paper document and the unique assignment of the first identifier to the second identifier, which is linked (inseparably) to the electronic document. This method thus allows further use of the paper document, which is used traditionally and which authorizes a certain action, in particular further use of a traditional paper prescription with which the issuer (e.g., the doctor) prescribes certain drugs and thus authorizes their transfer to the customer (patient). This makes it possible for prescription drug packages to be output to a pharmacy with a drug dispensing terminal, which is linked to an automated drug storage system and also permits remote control of this output by a pharmacist working at a remote location. This method ensures a legally reliable method of dispensing prescription drugs via a drug dispensing terminal by using a qualified electronic signature and by ensuring that the drug will not be dispensed to the customer until after the prescription has been signed, and doing so by a unique assignment of the first identifier applied to the prescription to a second identifier, which is linked to the signed electronic document.

A preferred embodiment of a method described herein is characterized in that the first identifier includes an alphanumeric code, and the second identifier is uniquely assigned to the first identifier and is linked to the electronic document by the fact that a file name of the electronic document comprising the alphanumeric code is used as the second identifier. This simplifies the assignment of the electronic documents in the secured memory to the paper documents carrying the first identifier.

A preferred refinement of the method is characterized in that the electronic image of the paper document is generated after the first identifier has been permanently applied to the paper document, whereby the electronic image from which the electronic document is generated includes an image of at least one part of the paper document with the first identifier applied to it. This procedure increases the security of the assignment of the identifier to the paper document because with this method, it possible to recognize, based on the image of the electronic document, which is being displayed at the second remote location of the person authorized to sign, whether the first identifier has been correctly applied to the paper document. Also, it readily permits a comparison of the first identifier, if it is legible in the image, with the second identifier, for example, the file name that has been linked to the electronic document.

Before applying the first unique identifier to the paper document, the paper document may be inserted into a receptacle and then the access to the paper document may be blocked, such that, if the method is terminated before the action has been carried out or if only a portion of the action has been carried out, then access to the paper document for retrieval is enabled again. Otherwise, after complete or partial execution of the action, the paper document is deposited in a filing system that is secured against unauthorized access. This ensures that the paper document cannot be reused to trigger the authorized action if this has already been done once. If the action has been carried out completely, for example, if the drugs identified on the prescription have been completely dispensed, the paper document (prescription) is retained and secured against unauthorized access.

In the event of partial execution of the action of the paper document, for example, in the case of partial dispensing of the drugs identified and the prescription form, then this portion of the action that has been carried out, for example, the drugs that have been dispensed, will be printed on the paper document and the paper document (prescription) will be retained and secured against unauthorized access (in an another method, the paper document could also be retrieved again after printing that part of the action that has been executed). Finally, the paper document can also be retrieved again if the action has not been carried out (even partially). That is, the method has been terminated before the action was carried out.

The paper document may be provided with an imprint characterizing complete execution of the action and/or the part of the action that has been executed and this is done before the paper document is placed in the file storage system where it is secured against unauthorized access. For example, a prescription may be provided with an imprint characterizing complete dispensing of the drugs identified on the prescription.

The paper document may contain information defining the type of authorized action. In this case, a first electronic image of the paper document is created. For example, the paper document may be scanned and converted to a graphic file. Based on the first electronic image, the type of action defined by the information is determined. There is a determination of whether it is possible to carry out the defined type of action (or even just a part of the action) at the first location. Only if this is the case is the method continued, wherein the electronic image, from which the electronic document is generated, is a second electronic image. Therefore, a first image of the paper document is preferably created first, so that it is possible on this basis to check on whether the type of action defined therein can be carried out. After applying the first identifier to the paper document, a second electronic image is generated and is used further for the purpose of the signature. In this refinement, it is possible to avoid printing out the paper document in case it is impossible to carry out the authorized action, which is characterized on the paper document. For example, dispensing the medications identified in the document may be impossible in certain situations.

Based on the first electronic image, the type of action defined by the information is determined by ascertaining the information by means of a text recognition program and ascertaining automatically whether it is possible to execute the defined type of action defined there. In the case of use of a prescription for automated output of drugs, this means that first there is a determination carried out automatically of which drugs are identified on the prescription and then there is a determination of whether it is possible to dispense these drugs.

The type of action defined by the information on the prescription may be determined based on the first electronic image. The image and the information about the actions to be carried out is displayed on the output device (for example, display screen) of the data processing system of the authorized person located at the second site. Depending on the result of the former, the authorized person stipulates the defined type of action by corresponding entries as a function thereof. In the case of a prescription for a drug output, this means that, on the one hand, the first electronic image of the prescription is displayed on the display screen of the pharmacist working at a remote site, while on the other hand, there is also a display of which drugs are on hand and can be dispensed at the first location. The pharmacist compares this information and then enables the output of the drug packages in stock. In the event that the drug identified on the prescription is not in stock, the pharmacist may optionally also enable an alternative drug to be dispensed.

The paper document may be a drug prescription and the authorized action is dispensing of drug packages identified on the prescription to a customer at the first location by a dispensing machine, wherein the image of the electronic document is displayed at the second remote location for the pharmacist as the person authorized to sign. A “pharmacist” here should also be understood to refer to any other person authorized to sign a prescription.

In a method described herein:

a1) The first electronic image of the prescription may be generated by the photographic device at the first location and displayed on the output device of the data processing system belonging to the pharmacist at the second remote location.
a2) A check may be carried out as to whether it is possible to dispense the drug packages identified on the prescription by an automatic dispenser located in the first location.
a3) If this dispensing is possible, conveyance of the drug packages to be dispensed to an output tray is instructed by a corresponding input by the pharmacist, while the access of the customer to the output tray is blocked.
a4) By means of detection devices at the first location, a check is carried out as to whether additional prerequisites for dispensing the drug packages have been met.
a5) If the additional prerequisites have been met, the first unique identifier is applied to the prescription by the application device and the second electronic image of the prescription is generated by the photographic device.
a6) The electronic document is generated from the second electronic image and is linked to the second identifier.

Access of the customer to the output tray is enabled. The dispensing security may be further increased by enabling the customer to access the output tray only when additional prerequisites for output of the drug packages have been met and the electronic signature has been executed.

An image of the drug packages conveyed into the output tray may be generated by means of a camera aimed at the output tray, and then, as an additional prerequisite for dispensing the drug packages, a check may be carried out based on the image to ascertain whether the correct drug packages are in the output tray. This refinement increases the security in dispensing because, before enabling access to the drug packages, the pharmacist is able to perform a final check on whether the automatic dispenser has retrieved the correct drug packages.

A request for a payment process may be output to the customer, and as additional prerequisites for dispensing the drug packages, there is a check on the success of the payment process. Access to the output tray here is advantageously also made dependent upon a successful payment process.

A check may be carried out to ascertain whether (after access has been enabled) all drug packages have been retrieved completely from the output tray that has been released, such that, if this is not the case, a signal is output to the customer, instructing the customer to be sure that all drug packages have been retrieved completely.

A check may be carried out of whether all drug packages have been retrieved completely from the output tray that has been released. If this is the case, the paper document is provided with an imprint indicating that the authorized action has been completed. Otherwise, the paper document may be provided with an imprint indicating that the retrieval of drug packages has not been complete. The drug packages not retrieved are then conveyed back into the drug storage area, for example. This also makes it possible to document incomplete retrieval of drug packages withdrawn from storage, which can also be correlated with the correct prescription.

### BRIEF DESCRIPTION OF THE DRAWINGS

The present disclosure is explained in greater detail below on the basis of a preferred exemplary embodiment, as illustrated in the drawings, in which:

FIG. 1 illustrates a schematic diagram of the devices that are linked together and used to implement the method according to the disclosure.

FIGS. 2A and 2B illustrate schematic diagrams of the process flows of a preferred embodiment of the method according to the disclosure.

### DETAILED DESCRIPTION

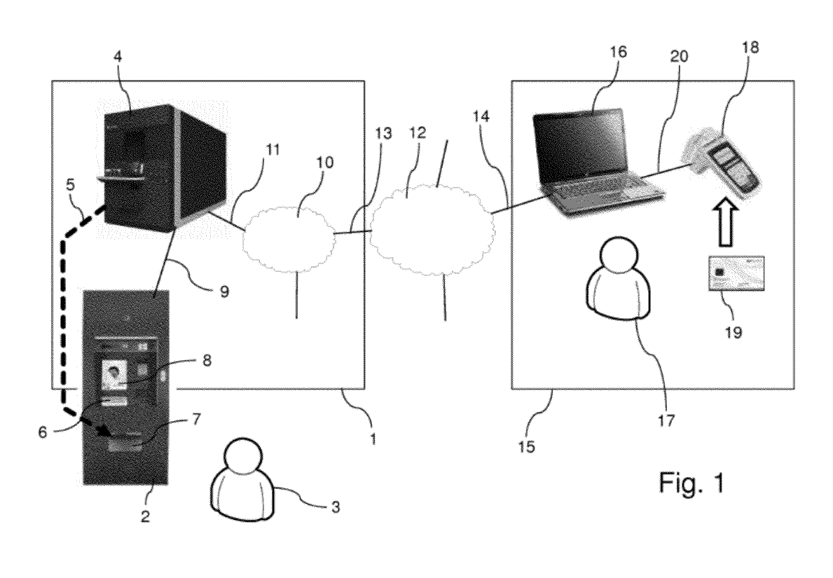

FIG. 1 illustrates a schematic diagram of the equipment used to implement the method according to the disclosure. One location has the facilities 1 of a pharmacy, which is represented by a rectangle. A drug output terminal 2 is arranged in such a way that it is accessible from the front side even when the facilities 1 of the pharmacy are closed. The back side of the drug dispensing terminal is situated inside the facilities 1 of the pharmacy. This type of arrangement of the terminal 2 is indicated in FIG. 1, where the terminal 2 is represented on the bordering line 1. The terminal 2, for example, may be set up in a foyer of a pharmacy. A customer 3 wanting to receive a drug, in particular a prescription drug, may then step in front of the terminal 2. The terminal 2 includes all the devices necessary for the customer 3 to have a dialogue with a pharmacist, to present a prescription, to pay the required amount and to retrieve the drug packages. In particular, the terminal 2 includes an output tray 7, which is closed by a lockable door (for example, a flap or rolling shutters), into which the drug packages to be dispensed can be conveyed and from which the customer 3 can retrieve the desired drug packages (after opening the door). This drug dispensing terminal 2 also includes an input shaft 6 for prescriptions, namely so that the customer 3 can insert a prescription. The terminal 2 also has a call button for initiating a desired drug dispensing transaction, a touchscreen 8 for display of requests to the customer and for input by the customer as well as a microphone, a loudspeaker and a camera for having a dialogue with the pharmacist. Finally, the terminal 2 has payment devices such as, for example, a coin slot, a currency slot, a card reader with a PIN input field, a receipt printer and a coin return.

The interior of the terminal contains additional components that are not accessible for the customer and include a data processing unit (server) for the terminal 2, a prescription scanner/printer combination, a collection tray for prescriptions that have been input as well as payment devices such as coin validators, coil collecting containers, change containers and currency validators. The terminal 2 has a solid metal housing. Access to the interior is secured by a lock on the back, and the lock is usually in the possession of the pharmacist. Access to the interior of the housing from the front side is impossible.

The server contained in the terminal 2 is linked via an in-house network to additional components of the terminal and to an automatic drug order-picking system, where the length between the terminal 2 and the automatic order picking system 4 is represented by the line 9 in FIG. 1. The connection between the terminal 2 and the automatic order picking system 4 is usually established by a cable installed in the pharmacy between a router for the automatic order picking system 4 and a switch on the terminal. Access to this network of the customer terminal from the outside is possible only within the context of the rules stored in a firewall in the router in the automatic order picking system 4 (for example, by means of a secured connection or a VPN).

The automatic order picking system 4 has storage shelves for storing drug packages whereby the storage shelves are arranged inside a housing, for example, so that a narrow lane is formed between them. A shelf operating unit, which can deposit packages on the shelves and retrieve packages from the shelves, moves in the lane between the storage shelves. During the periods of time when the pharmacy is open, the automatic order-picking system serves to deposit medication packages on the shelves and retrieve them from the shelves, such that the drug packages that are stored or dispensed are usually conveyed to an output site in the community pharmacy office. The shelf operating unit, which retrieves the packages from the shelves, then throws them in a discharge chute, for example, by means of which they are then conveyed by chutes/slides or conveyor belts to the dispensing location in the community pharmacy office.

In the times when the pharmacy is not occupied by employees, the automatic order-picking system 4 is linked by means of a conveyor system 5 to the drug output terminal 2 for implementing the method according to the disclosure. The shelf operating unit of the automatic order-picking system 4 deposits packages on the conveyor device 5, which ultimately conveys the packages to the output tray 7 of the terminal 2. The conveyor device 5 here includes conveyor belts and/or slides or other devices based on pneumatic tube technology, for example. The automatic order-picking system 4 has a control computer. All the components of the automatic order-picking system 4 are in turn linked to one another by a system network, and this system network of the automatic order-picking system 4 may be linked to an existing computer network 10 in the pharmacy by means of a line 11. Various computers, which are set up in the pharmacy, for example, may be connected to the pharmacy network 10.

The pharmacy network is in turn connected to the Internet 12 by a communication line 13, which includes the public telephone network, for example. FIG. 1 shows on the right side a box 15 indicating a home workstation of a pharmacist 17. That is the location of a computer 16, which is also connected to the Internet 12 by the line 14. In addition, the computer 16 is connected to a card reader by a connection 20. A signature card 19 can be inserted into the card reader 18. Other home workstations for various pharmacists in different locations may of course also be connected to the computer network 10 of the pharmacy or to the system network of the automatic order-picking system 4 via the Internet 12. Instead of a link via the Internet 12, a connection via any other bidirectional data communication link is also conceivable. For example, the computer 16 may also be connected to the network 10 of the pharmacy or to the system network of automatic order-picking system 4 via a mobile wireless connection.

Various programs that serve to carry out certain steps of the method according to the disclosure run on the data processing units of the various components of the system shown in FIG. 1, namely the data processing devices of the drug output terminal 2, the automatic order-picking system 4 and the computer 16. The data processing device of the terminal 2, which is known as the server, has a software component which is referred to as an “application server” and takes over control of the hardware components contained in the terminal 2 and control of the entire process and supplies a surface for management and maintenance of the system by the pharmacist from his computer over a linked-in web server. Furthermore, there is also a user interface by means of which a customer 3 operates the terminal 2. A database serves to store all the data on the procedure. Protocols, log files and signed prescriptions are stored on one or more hard drives of the server. A software component makes it possible to set up a video conference between the terminal 2 and the computer 16 of the pharmacist 17. In addition, the server also contains software components for increasing data security, such as a firewall, for example. The computer of the automatic order-picking system 4 contains the control software and management software for controlling the components of the automatic order-picking system and for management of the warehouse. In addition, the computer also contains the communication hardware and software required for establishing the aforementioned connections. The computer 16 at the pharmacist's home workstation also has, in addition to the operating system and various application programs, a pharmacist application program, which the pharmacist can use to control and monitor the drug output and the sales process. Software components that are used for the electronic signature and the video conference are embedded in this pharmacist application program. The computer 16 also has the card reader that is required for communicating with the card reader 18. The computer 16 has signature software for generating and checking a qualified electronic signature. The entire signature process is monitored exclusively by the signature software and includes the steps of opening the file to be signed, guiding the user through the signature process and the display of the contents to be signed, the control of the card reader, the application of the signature and the storage of the signed file.

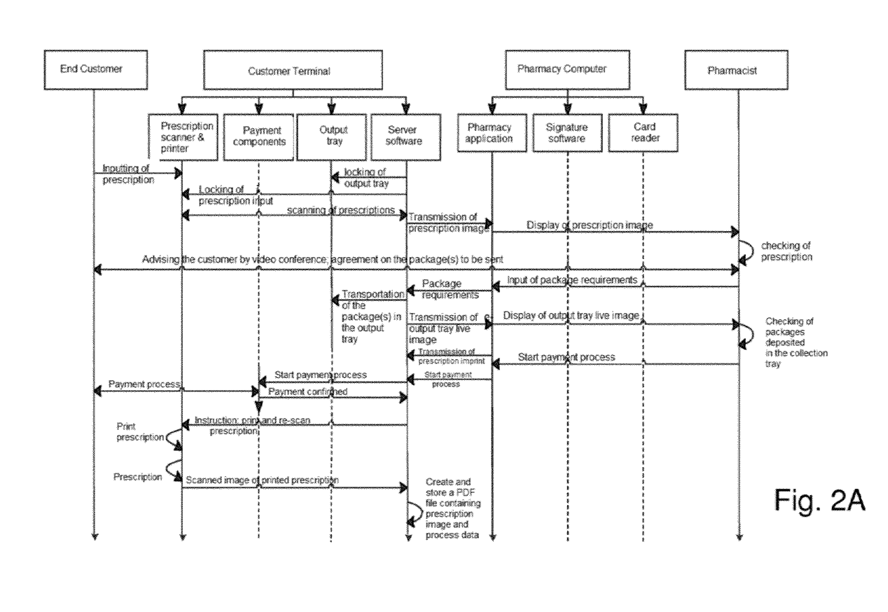

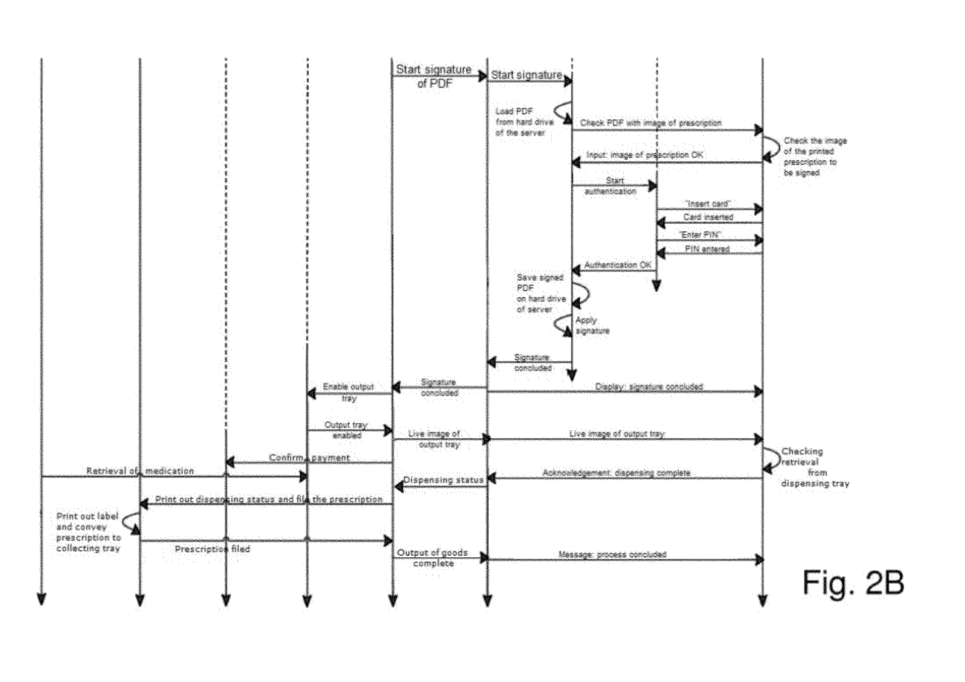

With reference to FIGS. 2A and 2B, a preferred method for output of prescription drugs via the remotely controlled drug dispensing terminal 2 is described below.

This method begins when the customer 3 stands in front of the terminal 2 and inserting his prescription into the intake slot 6 for prescriptions, whereupon the prescription goes to a prescription scanner. Immediately after input of the prescription, access to the intake slot 6 is locked, so that the customer 3 cannot withdraw the prescription without the consent of the pharmacist 17. Furthermore, the drug output tray 7 of the terminal 2 is locked. Any packages or other objects still present in the output tray are removed from the tray 7.

The prescription is scanned with control by the server software, so that a first electronic image of the prescription is generated. The electronic image of the prescription is then transmitted over the data communication link described above to the pharmacist application running on the computer 16, and then the image is displayed on the screen of the computer 16, so the pharmacist 17 can examine the prescription displayed there.

The pharmacist 17 contacts the customer 3 by means of the video conference equipment (conference software and video cameras, display screens, microphones and loudspeakers), whereupon the pharmacist advises the customer and they reach an agreement about the drug packages to be dispensed. After the pharmacist 17 has determined, on the basis of the prescription and in consultation with the customer 3, exactly which drug packages are to be dispensed, the pharmacist then enters the selection of the drug packages to be dispensed into the pharmacist application. The pharmacist can then check on the availability of the desired drug packages based on information transmitted to the pharmacist by the automatic order-picking system 4, so that the pharmacist requests only those drug packages that are also in stock in the automatic order-picking system 4. The pharmacist application then transmits a request for the packages to be output to the automatic order-picking system 4 and the server software of the terminal 2. The latter then ensure that the requested packages are conveyed to the output tray 7 of the terminal 2. If multiple drug packages are to be dispensed, the method of transmitting the packages to be dispensed by the pharmacist to the pharmacist application, forwarding of the information to the automatic order-picking system 4 and conveying the package to the output tray 7 can also be carried out step by step, package for package.

After conclusion of the conveyance of the drug packages to be dispensed (in another method described herein, this may also be done in advance), a live image of the drug packages sitting on the output tray is transmitted to the pharmacist, who then carries out a check of what has been dispensed, based on this image. Instead of one live image, a plurality of live images from various perspectives may also be transmitted to the pharmacist application and displayed on the screen of the pharmacist's computer 16, either simultaneously or electively.

As soon as the pharmacist is convinced that the correct drug packages have been placed in the output tray 7 and that payment for the drugs is required, the pharmacist enables the payment process by instructing the customer via video conference to pay for the drug packages. The payment process may then be carried out by means of coin or currency input or by entering a debit card or credit card, including entering a PIN. As soon as payment of the required amounts has been confirmed by the payment components, the server software instructs the prescription printer to print out the prescription with information about the dispensing of the drug packages and with a first unique identifier. The identifier may be, for example, a unique code (for example, a numeric code or an alphanumeric code).

As soon as the prescription has been printed with the information, a second electronic image of the prescription is generated with the help of the prescription scanner. An electronic document, for example, a PDF file, is generated from this second electronic image, wherein the electronic document is linked to a second identifier, which is uniquely assigned to the first identifier. For example, the file name of the electronic document may simply be used as the second identifier, and the file name in turn contains the code for the first identifier.

The further process will now be described with reference to FIG. 2B. After the PDF document obtained from the electronic image of the prescription printed with the first identifier has been saved on the hard drive of the server in terminal 2, the server of the pharmacist application signals that the prescription is ready for signing. The pharmacist application transmits to the signature software the file path under which the PDF document to be signed is saved as well as an additional file path under which the signed document is to be saved. The file names of the document that has been signed and of the unsigned document remain the same. The two versions differ only with regard to where they are saved on the hard drive of the server of the terminal 2. The file names may also be different and may be saved in the same directory. The files also need not necessarily be saved in the server of the terminal 2. They may also be saved in another secure memory.

After transmitting the memory location (file path), the PDF document is signed under the monitoring of the signature software. To do so, the PDF document is loaded from the hard drive of the server and displayed, whereupon the pharmacist verifies the image of the prescription.

After the pharmacist has input the fact that the image of the prescription is in order, an authentication is initiated. Within the context of this authentication, the pharmacist 17 may enter his signature card 19 into the reader 18. Then he is instructed to enter a PIN.

As soon as the correct PIN has been entered, the card reader signals that the authentication is in order, whereupon the signature software executes the signature and saves the signed PDF file at the predetermined location (directory according to file path) on the hard drive of the server. Then the signature software notifies the pharmacist application of the conclusion of the signature.

The pharmacist application notifies the server of the conclusion of the signature whereupon the server unlocks the output tray 7, e.g., enables it. The customer receives the display that he/she can retrieve the medication. As soon as the message “Output tray enabled” has been output to the server software, this transmits a live image of the output tray 7 to the pharmacist application, which ensures a display of the live image of the output tray 7 on the display device of the pharmacist's computer 16. The server software then confirms the payment to the payment components. In addition, the customer 3 retrieves the medication from the output tray 7 and the pharmacist 17 observes this on the basis of the transmitted live image whereupon he enters a corresponding acknowledgment to the pharmacist application after all of the drug packages have been retrieved from the output tray. This dispensing status is transmitted from the pharmacist application to the server software. The server software then ensures that the dispensing status is printed on the prescription form and the printed prescription is securely stored. After printing, the prescription is conveyed to the prescription filing tray. Conclusion of the transport is displayed for the server software which then forwards the message “Dispensing of goods completed” to the pharmacist application, which then displays a corresponding message to the pharmacist indicating that the procedure is concluded.

There are a number of routines that may react to different errors or other conditions that differ from the normal procedure described above. For example, it is possible that not all of the drug packages prescribed on the prescription form are available in the automatic order-picking system 4 or can be substituted by drug packages available there. This information is displayed for the pharmacist, whereupon he either terminates the sale and the prescription may be returned to the customer, or the drug packages that are available may be dispensed. Then the procedure described above is modified slightly if the pharmacist decides to dispense only some of the drug packages as printed on the prescription form. An additional section identifying the prescription items not dispensed is included in the signed PDF file. In addition, the signed PDF file contains the notation “Subsequent dispensing by manual signature on the paper prescription form.” “Partial amount” is printed as the dispensing status on the paper prescription form before the prescription is filed. Then if the remaining drugs from the prescription are dispensed later, this may be noted manually on the paper prescription form and signed in this embodiment.

Another case is when the procedure is terminated before payment takes place. If the sale of a prescription medication is terminated before conclusion of the payment process, that is, before the prescription is printed, then the prescription can be returned to the customer. If cash has already been paid out, it is returned to the customer, and any card payment transactions that have been initiated are terminated. The drug packages already conveyed into the output tray 7 are emptied out of the output tray and can be returned to storage in the automatic order-picking system 4, for example.

There may also be a case in which the signature is not successful. In this case, first the signature process is repeated. If the signature ultimately fails, the pharmacist application will then signal to the computer 16 of the server software that the sales transaction is being terminated. Any cash that has been paid is returned to the customer, and a card payment, if any, is canceled. However, the prescription now cannot be returned to the customer because it has already been printed out. Instead of that it is printed out with the status “no signature” and conveyed to the filing tray. Meanwhile the pharmacist can inform the customer by video conference about how to proceed further. If the output tray has already been filled, it is emptied.

A number of alternatives are conceivable within the scope of the claimed processes instead of the procedures described above which are currently preferred.

1. A method for triggering an action of a device authorized by a paper document, wherein the action presupposes a unique assignment of a signature to the paper document, the method comprising:
a) inserting the paper document into a receptacle at a first location where the action is to be carried out, permanently applying a first unique identifier to the paper document by an application device and generating an electronic image of the paper document, wherein a second identifier is uniquely assigned to the first identifier and an electronic document is generated from the electronic image and is linked to the second identifier,
b) displaying an image of the electronic document on an output device of a data processing unit of a person authorized for signing, said data processing unit being located at a second location which is remote, and electronically signing the electronic document by the authorized person by means of a predefined input into an input device,
c) storing the signed electronic document, which is linked to the second identifier, in a secured memory, and
d) carrying out the authorized action by the device when the signed electronic document has been saved.

2. The method of claim 1, wherein the first identifier comprises an alphanumeric code, wherein the second identifier is uniquely assigned to the first identifier and is linked to the electronic document in that a file name of the electronic document containing the alphanumeric code is used as the second identifier.

3. The method of claim 1, wherein the electronic image of the paper document is generated after the first identifier has been permanently applied to the paper document, wherein the electronic image from which the electronic document is generated comprises an image of at least a portion of the paper document with the first identifier applied.

4. The method of claim 1, further comprising:
before applying the first unique identifier to the paper document, inserting the paper document into a receptacle and locking access to the paper document,
if the method is terminated before the authorized action is carried out or if only part of the authorized action has been carried out, enabling access to the paper document for the retrieval,
after the action has been carried out completely or partially, storing the paper document is stored in a storage site that is secured against unauthorized access.

5. The method of claim 4, wherein the paper document is provided with an imprint indicating that all or part of the action has been carried out before the paper document is stored in the storage site that is secured from unauthorized access.

6. The method of claim 1, wherein the paper document contains information which defines the type of authorized action, and wherein the method further comprises:
creating a first electronic image of the paper document,
determining, based on the first electronic image, the type of action defined by the information,
determining whether carrying out the defined type of action at the first location is possible, and
performing step a) if it is determined that carrying out the defined type of action at the first location is possible,
wherein the electronic image created in step a) and from which the electronic document is generated is a second electronic image.

7. The method of claim 6, wherein determining the type of action is based on the first electronic image and comprises ascertaining the information by means of a text recognition program and automatically determining whether carrying out the defined type of action is possible.

8. The method of claim 6, wherein, based on the first electronic image, the type of action defined by the information is determined by displaying, for the authorized person, the image and the information about actions that can be executed on the output device of the data processing system located at the second location, and said authorized person stipulates the defined type of action through corresponding inputs as a function thereof.

9. The method of claim 8, wherein the paper document comprises a drug prescription form and the authorized action comprises dispensing of drug packages identified on the prescription to a customer at the first location by a dispensing device, wherein the image of the electronic document is displayed for a pharmacist as the person authorized for signing at the remote second location.

10. The method of claim 9, further comprising:
a1) generating the first electronic image of the prescription by the photographic device at the first location and displayed on the output device of the data processing system belonging to the pharmacist at the remote second location,
a2) carrying out a check as to whether it is possible to dispense the drug packages identified on the prescription by an automatic dispenser located in the first location,
a3) if this dispensing is possible, instructing conveyance of the drug packages to be dispensed to an output tray by a corresponding input by the pharmacist, while the access of the customer to the output tray is blocked,
a4) carrying out a check, by means of detection devices at the first location, of whether additional prerequisites for dispensing the drug packages have been met,
a5) if the additional prerequisites have been met, applying the first unique identifier to the prescription by the application device and generating the second electronic image of the prescription by the photographic device,
a6) generating the electronic document from the second electronic image and is linked to the second identifier, and
enabling the access of the customer to the output tray, wherein customer access the output tray is enabled when additional prerequisites for output of the drug packages have been met and the electronic signature has been executed, thereby increasing dispensing security.

11. The method of claim 10, wherein carrying out the check of whether additional prerequisites for dispensing the drug packages has been met comprises generating an image of the drug packages conveyed into the output tray by means of a camera aimed at the output tray, and carrying out a check, as an additional prerequisite for dispensing the drug packages, based on the image, to ascertain whether the correct drug packages are in the output tray.

12. The method of claim 10, further comprising outputting a request for a payment process to the customer and carrying out a check, as additional prerequisites for dispensing the drug packages, on whether the payment process has been successfully concluded.

13. The method of claim 10, further comprising carrying out a check on whether all the drug packages have been completely retrieved from the enabled output tray and outputting a signal to the customer if all the drug packages have not been completely retrieved from the enabled output tray.

14. The method of claim 10, further comprising carrying out a check on whether all drug packages have been completely retrieved from the enabled output tray, and if if all drug packages have been completely retrieved from the enabled output tray, providing the paper document with an imprint indicating that the authorized action has been completed, and if all drug packages have not been completely retrieved from the enabled output tray, providing the paper document with an imprint indicating that the retrieval of the drug packages has been incomplete.

15. A method for initiating an action of a device authorized by a paper document, the method comprising:
receiving the paper document into a receptacle at a first location, wherein the first location is where the action is performed;
permanently applying a first unique identifier to the paper document;
generating an electronic document based on the paper document;
associating a second identifier with the first identifier and the electronic document;
displaying, at a second location, remote from the first location, the electronic document to an authorized person;
receiving and applying to the electronic document, an electronic signature by the authorized person to generate a signed electronic document;
storing the signed electronic document in a secured memory; and
performing the action by the device upon storing the signed electronic document.

16. The method of claim 15, wherein the first identifier comprises an alphanumeric code, and wherein the second identifier comprises a file name of the electronic document, the file name containing the alphanumeric code.

17. The method of claim 15, further comprising:
locking access to the paper document;
if the method is terminated before the action is performed or if the entirety of the action has not been performed, enabling access to the paper document for retrieval;
upon performing the action, storing the paper document in a storage site, the storage site being secured against unauthorized access.

18. The method of claim 15, further comprising:
determining, based on the paper document, whether performing the action is possible.

19. The method of claim 15, wherein the paper document comprises a drug prescription form and the action comprises dispensing of drug packages identified on the prescription to a customer at the first location by a dispensing device.

20. A method for approving an action of a device authorized by a paper document, the method comprising:
receiving, from the device at a remote location, an electronic document, the electronic document being associated with an identifier;
displaying the electronic document;
receiving an electronic signature;
applying the electronic signature to the electronic document to create a signed electronic document; and
saving the signed electronic document, wherein saving the signed electronic document triggers performing the action by the device at the remote location.
